(19) United States
(12) Patent Application Publication
(10) Pub. No.: US 2024/0293784 A1
(43) Pub. Date: Sep. 5, 2024

(54) Thermo-induced stimuli-responsive membrane for leukocyte enrichment and application thereof

(71) Applicant: CHUNG YUAN CHRISTIAN UNIVERSITY, Tao-Yuan (TW)

(72) Inventor: Yung Chang, Tao-Yuan (TW)

(21) Appl. No.: 18/582,695

(22) Filed: Feb. 21, 2024

Related U.S. Application Data
(60) Provisional application No. 63/447,979, filed on Feb. 24, 2023.

(51) Int. Cl. B01D 71/40; A61B 5/15; B01D 69/14

(52) U.S. Cl. CPC B01D 71/401; A61B 5/150755; B01D 69/14

(57) ABSTRACT

A novel thermo-induced stimuli-responsive membrane for leukocyte enrichment and its application to white blood cells were disclosed. Specifically, the thermo-induced stimuli-responsive membrane for leukocyte enrichment comprises a layer coated on a porous substrate, and composition of the layer comprises at least one copolymer selected from one of group consisting of poly(acrylic acid-co-alkyl methacrylate), poly(N-alkyl acrylamide-co-alkyl methacrylate) and their mixture. In particular, the time for white blood cells recovery is within 1 hour, so as to obtain fresh and high purity white blood cells by using the novel thermo-induced stimuli-responsive membrane.

### TECHNICAL FIELD

The invention relates to a thermo-induced stimuli-responsive membrane for leukocyte enrichment and its application to white blood cells recovery. Specifically, the thermo-induced stimuli-responsive membrane comprises a layer coated on a porous substrate, and composition of the layer comprises at least one copolymer selected from one of group consisting of poly(acrylic acid-co-alkyl methacrylate), poly(N-alkyl acrylamide-co-alkyl methacrylate) and their mixture.

### BACKGROUND

In general, leukocytes are separated from blood samples by gradient centrifugation method. This method is well known and widely used in blood treatment and transfusion. In addition, in order to avoid blood coagulation or platelet activation, usually a blood sample should be added with an anti-coagulant agent or the surface of the medical device should be further surface-treated for anti-coagulation. Another known material for blood treatment is a zwitterionic polymer, such as polysulfobetaine, due to its bio-antifouling property.

As so far, it is still difficult to effectively recover leukocytes from blood samples by the known methods. The gradient centrifugation method for recovering leukocytes gives low efficacy and low leukocyte purity due to its long operation time and complicated process. Bio-antifouling materials, such as polysulfobetaine, can avoid leukocytes to attach on medical device surface but not recover leukocytes from the blood sample. Accordingly, there is a continuing need to develop an efficient solution or method for recovering white blood cells and to achieve the goals of safety blood treatment and transfusion.

### SUMMARY OF THE INVENTION

In view of the above background of the invention and to meet the requirement of the medical industries. The objective of the invention is to provide a novel white blood cells or leukocytes recovering technology.

In the specification, definition and meaning of leukocytes is the same as white blood cells.

In one aspect, a thermo-induced stimuli-responsive membrane was disclosed. Specifically, the thermo-induced stimuli-responsive membrane is applied to enrich leukocytes and/or recover white blood cells from a blood sample.

Typically, the thermo-induced stimuli-responsive membrane for leukocyte enrichment comprises a layer and a porous substrate, and the layer is coated on surface of the porous substrate. Hence, the thermo-induced stimuli-responsive membrane for leukocyte enrichment comprises the layer coated on the porous substrate, and composition of the layer comprises at least one copolymer selected from one of group consisting of poly(acrylic acid-co-alkyl methacrylate), poly(N-alkyl acrylamide-co-alkyl methacrylate) and their mixture. Preferably, alkyl methacrylate is butyl methacrylate (BMA) and N-alkyl acrylamide is N-isopropylacrylamide (NIPAAm).

In one embodiment, the thermo-induced stimuli-responsive membrane for leukocyte enrichment has a coating density more than 0.02 mg/cm² on its surface.

In one preferred embodiment, the layer has characteristic peaks in XPS spectrum at following binding energy: 285±0.2 eV, 285.94±0.2 eV, 288.11±0.2 eV, 289.26±0.2 eV, 532.22±0.2 eV and 533.52±0.2 eV. Preferably, the layer contains following functional groups: amide group, ester group, carboxylic acid group or its combinations. More preferably, the layer has 77-96% of carbon, 5-15% of oxygen and 0-10% nitrogen based on XPS analysis. Most preferably, the layer has 82-87% of carbon, 10-15% of oxygen and 2-4% nitrogen based on XPS analysis.

In another embodiment, the porous substrate comprises PP, PTFE, PVDF, PET, PBT, PU, nylon, PE, PS, ceramic or rayon.

In another aspect, the thermo-induced stimuli-responsive membrane for leukocyte enrichment is a novel thermo responsive membrane and capable of capturing leukocytes from blood samples or bio-fluids at 20-40° C. onto the thermo responsive membrane and then releasing the leukocytes from the thermo responsive membrane at 0-10° C. Therefore, the thermo responsive membrane is excellent and effective for separating and recovering leukocytes from blood samples or bio-fluids.

In one embodiment, the thermo responsive membrane for leukocyte enrichment comprises a coating layer formed from a copolymer mixture. The copolymer mixture contains at least one copolymer for capturing leukocytes and at least one thermo-responsive copolymer. Preferably, the copolymer for capturing leukocytes comprises poly(acrylic acid-co-alkyl methacrylate) and the thermo-responsive copolymer comprises poly(N-alkyl acrylamide-co-alkyl methacrylate).

In another aspect, the invention provides a method for recovering white blood cells. The method for recovering white blood cells is to use the aforementioned thermo-induced stimuli-responsive membrane or a leukocyte separation bed for selective attaching or detaching white blood cells at different temperature, so as to achieve purpose of recovery of the white blood cells.

Generally, the operation time of the method for recovering white blood cells is within 1 hour. In contrast, the known leukocytes recovering technology by gradient centrifugation takes at least 2 hours. As a result, the invented method for recovering white blood cells based on the thermo-induced stimuli-responsive membrane or the leukocyte separation bed is superior to the known leukocytes recovering technology by gradient centrifugation. Furthermore, the white blood cells recovered by using the invented method are fresh and have higher purity due to short operation time. This is also one of the advantages of the invented method.

Specifically, the method comprises following steps.
Step 1: filter a blood sample comprises white blood cells through a filter that comprises at least 12 layers of a thermo-induced stimuli-responsive membrane or a column that comprises a leukocyte separation bed by gravity at 20-40° C. for attaching white blood cells from the blood sample onto the thermo-induced stimuli-responsive membrane or the leukocyte separation bed.
Step 2: incubate the filter or column at 0-10° C. for 10 minutes at least for detaching the white blood cells from the thermo-induced stimuli-responsive membrane or the leukocyte separation bed.
Step 3: elute the filter or column with a liquid by gravity at 0-10° C. to recover the white blood cells from the blood sample and obtain a white blood cells concentrate that has a concentration of the white blood cells more than 2.0×10⁷ cells/ml.

In one embodiment, the thermo-induced stimuli-responsive membrane comprises a layer and a porous substrate, and the layer is coated on surface of the porous substrate. Hence, the thermo-induced stimuli-responsive membrane for leukocyte enrichment comprises the layer coated on the porous substrate, and composition of the layer comprises at least one copolymer selected from one of group consisting of poly(acrylic acid-co-alkyl methacrylate), poly(N-alkyl acrylamide-co-alkyl methacrylate) and their mixture. Preferably, alkyl methacrylate is butyl methacrylate (BMA) and N-alkyl acrylamide is N-isopropylacrylamide (NIPAAm).

In one embodiment, the thermo-induced stimuli-responsive membrane has a coating density more than 0.02 mg/cm² on its surface.

In one preferred embodiment, the layer has characteristic peaks in XPS spectrum at following binding energy: 285±0.2 eV, 285.94±0.2 eV, 288.11±0.2 eV, 289.26±0.2 eV, 532.22±0.2 eV and 533.52±0.2 eV. Preferably, the layer contains following functional groups: amide group, ester group carboxylic acid group or its combinations. More preferably, the layer has 77-96% of carbon, 5-15% of oxygen and 0-10% nitrogen based on XPS analysis. Most preferably, the layer has 82-87% of carbon, 10-15% of oxygen and 2-4% nitrogen based on XPS analysis.

In another embodiment, the porous substrate comprises PP, PTFE, PVDF, PET, PBT, PU, nylon, PE, PS, ceramic or rayon.

In another embodiment, the leukocyte separation bed formed from poly(acrylic acid) hydrogel, poly(N-isopropylacrylamide) hydrogel or a hydrogel synthesized from a mixture of acrylic acid and N-isopropylacrylamide.

In still another embodiment, the mixture of acrylic acid and N-isopropylacrylamide has a weight ratio of acrylic acid to N-isopropylacrylamide being 4-9. More preferably, the weight ratio of acrylic acid to N-isopropylacrylamide is 6-9.

In one representative embodiment, the method for recovering white blood cells from a blood sample comprises following steps. Place the assembled syringe and filter assembled the thermo-induced stimuli-responsive membrane on top of an open collection tube. Collect 10 ml samples of freshly collected blood from donors are treated with general anticoagulant such as CPDA-1, EDTA, sodium citrate or heparin. Allow the treated blood samples to drain via gravity through the syringe and filter into the collection tube. After filtration, prepare a 25 mm diameter syringe module. Add 5 ml of cold PBS solution maintained at 4° C. to the syringe module. Incubate the syringe module at 4° C. for 30 minutes. After incubation, place the filter with the syringe on a new 50 ml collection tube. Push the total 20 ml cold PBS through the filter to recover white blood cells attached to the thermo-induced stimuli-responsive membranes. Pipette up and down the syringe module to ensure through elution of leukocytes from the filter. The collected PBS solution was then analyzed with a Complete Blood Count (CBC) instrument (XN-1000, Sysmex).

In conclusion, the invented thermo-induced stimuli-responsive membrane is a thermo responsive membrane and capable of capturing leukocytes from blood samples or bio-fluids at 20-40° C. and then releasing the leukocytes at 0-10° C. Therefore, the thermo-induced stimuli-responsive membrane is excellent and effective for separating and recovering leukocytes from blood samples or bio-fluids. Moreover, the method for recovering white blood cells is to use the aforementioned thermo-induced stimuli-responsive membrane or a leukocyte separation bed for selective attaching or detaching white blood cells at different temperature, so as to achieve purpose of recovering the white blood cells from blood samples. In particular, the operation time of the method for recovering white blood cells is within 1 hour. Accordingly, the white blood cells recovered by using the invented thermo-induced stimuli-responsive membrane and method are fresh and have higher purity than the known leukocytes recovery technology.

### BRIEF DESCRIPTION OF THE DRAWINGS

FIG. 1 is a flow chart of the method for recovering white blood cells;

FIG. 2 is ¹H-NMR spectrum of poly(AA-co-BMA);

FIG. 3 is ¹H-NMR spectrum of poly(NIPAAm-co-BMA);

FIG. 4 is FTIR spectrums of poly(AA-co-BMA), poly(NIPAAm-co-BMA) and their mixture;

FIG. 5 is XPS spectrums of poly(AA-co-BMA), poly(NIPAAm-co-BMA) and their mixture;

FIG. 6 is a bar chart of coating density of poly(AA-co-BMA);

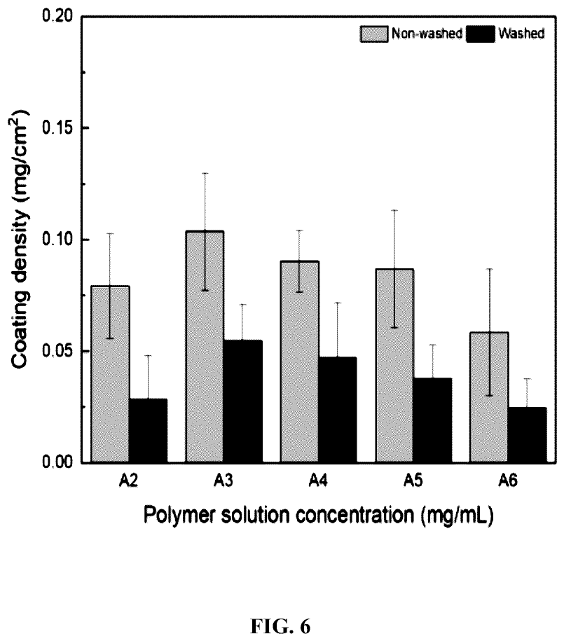

FIG. 7 is a bar chart of coating density of poly(NIPAAm-co-BMA);

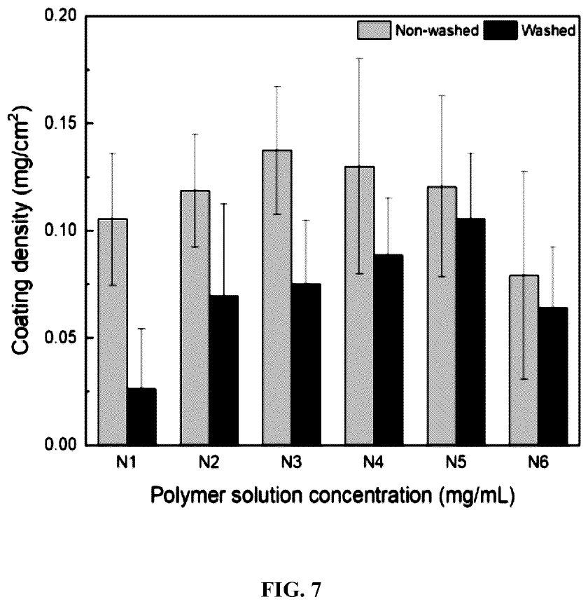

FIG. 8 is a plot of water contact angle of poly(AA-co-BMA) vs. time at 25° C.;

FIG. 9 is a plot of water contact angle of poly(NIPAAm-co-BMA) vs. time at 37° C.;

FIG. 10 is a plot of water contact angle of poly(NIPAAm-co-BMA) vs. time at 4° C.;

FIG. 11 is a plot of water contact angle of the copolymer mixture vs. time at 37° C.;

FIG. 12 is a plot of water contact angle of the copolymer mixture vs. time at 4° C.;

FIG. 13 is images of leukocytes adhesion on poly(AA-co-BMA) the at 37° C. and 4° C.;

FIG. 14 is images of leukocytes adhesion on the poly(NIPAAm-co-BMA) at 37° C. and 4° C.;

FIG. 15 is images of leukocytes adhesion on the copolymer mixtures at 37° C. and 4° C.;

FIG. 16 is images of leukocytes adhesion on the copolymer mixtures by DAPI and FITC staining at 37° C. and 4° C.;

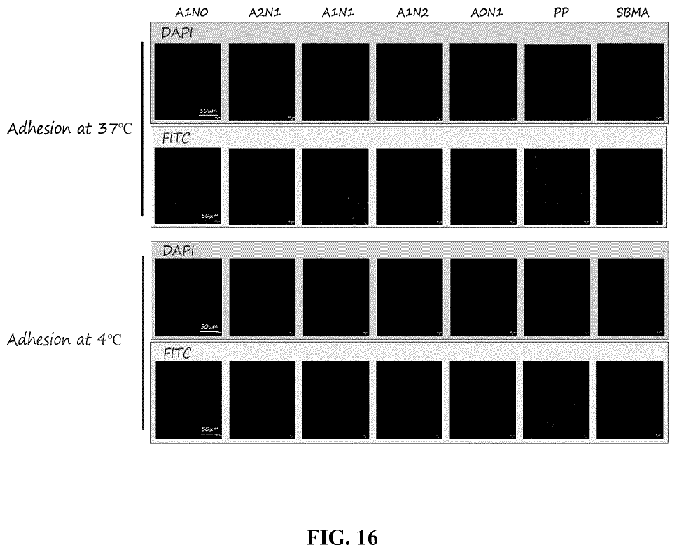

FIG. 17 is a bar chart of number of attached and detached leukocytes on the hydrogels;

FIG. 18 is a bar chart of hydrogel interface detached ratio;

FIG. 19 is a bar chart of leukocyte viability on the hydrogels; and

FIG. 20 is a bar chart of number of attached and detached leukocytes on the hydrogels at 37° C. and 4° C.

### EMBODIMENTS

In a first embodiment, the invention discloses a thermo-induced stimuli-responsive membrane for leukocyte enrichment. Specifically, the thermo-induced stimuli-responsive membrane is applied to enrich leukocytes and/or recover white blood cells from a bio-sample.

In one example of the first embodiment, the thermo-induced stimuli-responsive membrane for leukocyte enrichment comprises a layer coated on a porous substrate, and composition of the layer comprises at least one copolymer selected from one of group consisting of poly(acrylic acid-co-alkyl methacrylate), poly(N-alkyl acrylamide-co-alkyl methacrylate) and their mixture. Preferably, alkyl methacrylate is butyl methacrylate (BMA) and N-alkyl acrylamide is N-isopropylacrylamide (NIPAAm).

In one example of the first embodiment, the thermo-induced stimuli-responsive membrane has a coating density more than 0.02 mg/cm².

In one example of the first embodiment, the layer has characteristic peaks in XPS spectrum at following binding energy: 285±0.2 eV, 285.94±0.2 eV, 288.11±0.2 eV, 289.26±0.2 eV, 532.22±0.2 eV and 533.52±0.2 eV. Preferably, the layer contains following functional groups: amide group, ester group and carboxylic acid group. More preferably, the layer has 77-96% of carbon, 5-15% of oxygen and 0-10% nitrogen based on XPS analysis. Most preferably, the layer has 82-87% of carbon, 10-15% of oxygen and 2-4% nitrogen based on XPS analysis.

In another example of the first embodiment, the porous substrate comprises PP, PTFE, PVDF, PET, PBT, PU, nylon, PE, PS, ceramic or rayon.

In another example of the first embodiment, the poly(acrylic acid-co-alkyl methacrylate) has a molar ratio of acrylic acid to alkyl methacrylate being from 1.1 to 5. Preferably, the poly(acrylic acid-co-alkyl methacrylate) has a weight average molecule weight between 1 and 300 kDa. Preferably, the weight average molecule weight is between 60 and 90 kDa.

In another example of the first embodiment, the poly(N-alkyl acrylamide-co-alkyl methacrylate) has a molar ratio of N-alkyl acrylamide to alkyl methacrylate being from 1 to 6. Preferably, the poly(N-alkyl acrylamide-co-alkyl methacrylate) has a weight average molecule weight between 1 and 300 kDa. Preferably, the weight average molecule weight between 40 and 60 kDa.

In another example of the first embodiment, the mixture comprises 1-99 wt. % of poly(acrylic acid-co-butyl methacrylate) and 1-99 wt. % of poly(N-isopropylacrylamide-co-butyl methacrylate). Preferably, the mixture comprises 50±30 wt % of poly(acrylic acid-co-butyl methacrylate) and 50±30 wt % of poly(N-isopropylacrylamide-co-butyl methacrylate). More preferably, the mixture comprises 50±5 wt % of poly(acrylic acid-co-butyl methacrylate) and 50±5 wt % of poly(N-isopropylacrylamide-co-butyl methacrylate).

In a second embodiment, the invention provides a method for recovering white blood cells. The method comprises following steps.
Step 1: filter a blood sample comprises white blood cells through a filter that comprises at least 12 layers of a thermo-induced stimuli-responsive membrane or a column that comprises a leukocyte separation bed by gravity at 25-40° C. for attaching white blood cells from the blood sample onto the thermo-induced stimuli-responsive membrane or the leukocyte separation bed.
Step 2: incubate the filter or column at 0-10° C. for 10 minutes at least for detaching the white blood cells from the thermo-induced stimuli-responsive membrane or the leukocyte separation bed.
Step 3: elute the filter or column with a liquid by gravity at 0-10° C. to recover the white blood cells from the blood sample and obtain a white blood cells concentrate that has a concentration of the white blood cells more than 2.0×10⁷ cells/ml.

In one example of the second embodiment, the thermo-induced stimuli-responsive membrane comprises a layer coated on a porous substrate, and composition of the layer comprises at least one copolymer selected from one of group consisting of poly(acrylic acid-co-alkyl methacrylate), poly(N-alkyl acrylamide-co-alkyl methacrylate) and their mixture. Preferably, alkyl methacrylate is butyl methacrylate (BMA) and N-alkyl acrylamide is N-isopropylacrylamide (NIPAAm).

In one example of the second embodiment, the thermo-induced stimuli-responsive membrane has a coating density more than 0.02 mg/cm².

In one example of the second embodiment, the layer has characteristic peaks in XPS spectrum at following binding energy: 285±0.2 eV, 285.94±0.2 eV, 288.11±0.2 eV, 289.26±0.2 eV, 532.22±0.2 eV and 533.52±0.2 eV. Preferably, the layer contains following functional groups: amide group, ester group and carboxylic acid group. More preferably, the layer has 77-96% of carbon, 5-15% of oxygen and 0-10% nitrogen based on XPS analysis. Most preferably, the layer has 82-87% of carbon, 10-15% of oxygen and 2-4% nitrogen based on XPS analysis.

In another example of the second embodiment, the porous substrate comprises PP, PTFE, PVDF, PET, PBT, PU, nylon, PE, PS, ceramic or rayon.

In another example of the second embodiment, the poly(acrylic acid-co-alkyl methacrylate) has a molar ratio of acrylic acid to alkyl methacrylate being from 1.1 to 5. Preferably, the poly(acrylic acid-co-alkyl methacrylate) has a weight average molecule weight between 1 and 300 kDa. Preferably, the weight average molecule weight is between 60 and 90 kDa.

In another example of the second embodiment, the poly(N-alkyl acrylamide-co-alkyl methacrylate) has a molar ratio of N-alkyl acrylamide to alkyl methacrylate being from 1 to 6. Preferably, the poly(N-alkyl acrylamide-co-alkyl methacrylate) has a weight average molecule weight between 1 and 300 kDa. Preferably, the weight average molecule weight is between 40 and 60 kDa.

In another example of the second embodiment, the mixture comprises 1-99 wt. % of poly(acrylic acid-co-butyl methacrylate) and 1-99 wt. % of poly(N-isopropylacrylamide-co-butyl methacrylate). Preferably, the mixture comprises 50±30 wt % of poly(acrylic acid-co-butyl methacrylate) and 50±30 wt % of poly(N-isopropylacrylamide-co-butyl methacrylate). More preferably, the mixture comprises 50±5 wt % of poly(acrylic acid-co-butyl methacrylate) and 50±5 wt % of poly(N-isopropylacrylamide-co-butyl methacrylate).

In another example of the second embodiment, the leukocyte separation bed formed from poly(acrylic acid) hydrogel, poly(N-isopropylacrylamide) hydrogel or a hydrogel synthesized from a mixture of acrylic acid and N-isopropylacrylamide.

In another example of the second embodiment, the mixture of acrylic acid and N-isopropylacrylamide has a weight ratio of acrylic acid to N-isopropylacrylamide being 4-9. More preferably, the weight ratio of acrylic acid to N-isopropylacrylamide is 6-9.

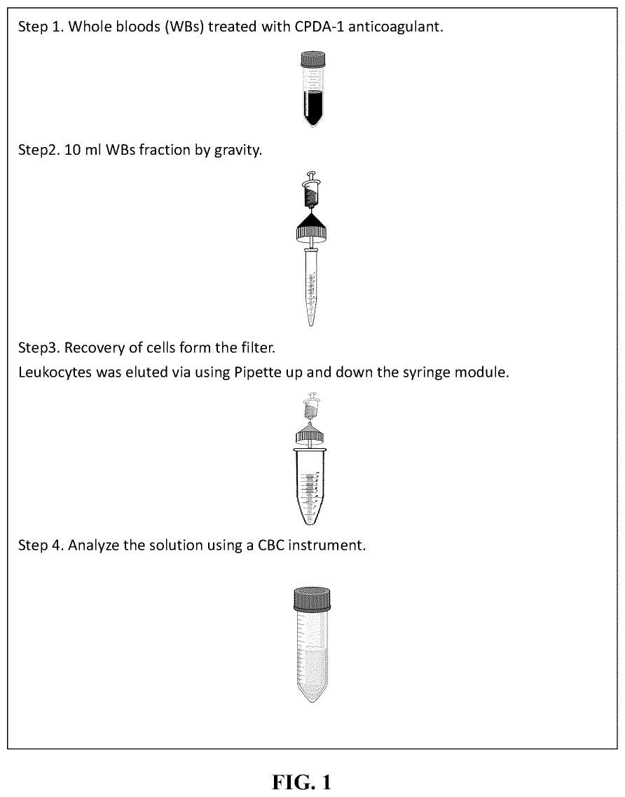

In one representative example of the second embodiment, please refer to FIG. 1, the method for recovering white blood cells from a blood sample comprises following steps. Attach the 10 ml syringe to the filter assembled the thermo-induced stimuli-responsive membranes or the leukocyte separation bed. Place the assembled syringe and the filter on top of an open 15 ml collection tube. Collect 10 ml samples of freshly collected blood from donors are treated with CPDA-1 anticoagulant. Allow the treated blood samples to drain/filter via gravity, through the filter into the collection tube. Recover first cells form the filter. After filtration, prepare a 25 mm diameter syringe module. Add 5 ml of cold PBS solution maintained at 4° C. to the syringe module. Incubate the syringe module at 4° C. for 30 minutes. After incubation, place the filter with the syringe on a new 50 ml collection tube. Push the total 20 ml cold PBS through the filter to recover white blood cells attached to the thermo-induced stimuli-responsive membranes or leukocyte separation bed. Elution of white blood cells/leukocytes is to pipette up and down the syringe module to ensure through elution of leukocytes from the filter. Perform an analysis to measure the number of recovering white blood cells. The complete solution was then analyzed with a Complete Blood Count (CBC) instrument (XN-1000, Sysmex).

### Synthesis of Poly(AA-Co-BMA)

The reaction scheme is shown in following.

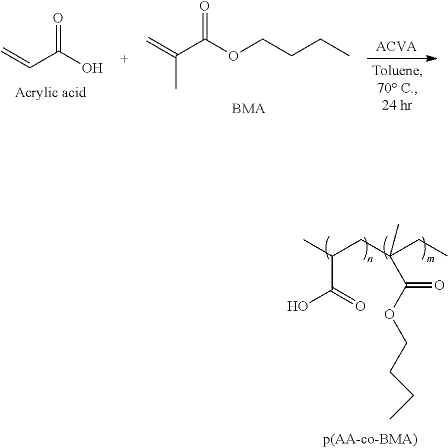

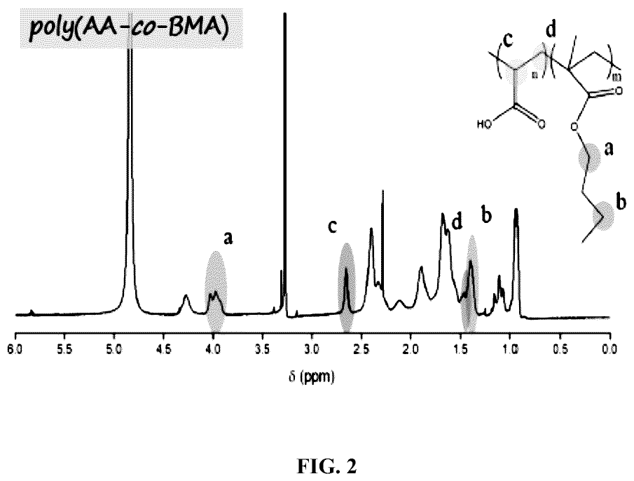

Acrylic acid and BMA were dissolved in toluene and then polymerized in the presence of 4,4′-azobis(4-cyanovaleric acid) (ACVA) at 70° C. for 24 hours. After purification, poly(AA-co-BMA) was obtained and analyzed by NMR. The NMR spectrum of poly(AA-co-BMA) was shown in FIG. 2.

### Synthesis of Poly(NIPAAm-Co-BMA)

The reaction scheme is shown in following.

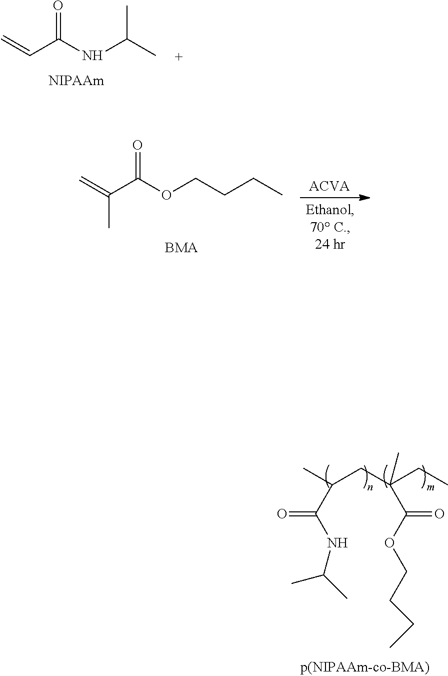

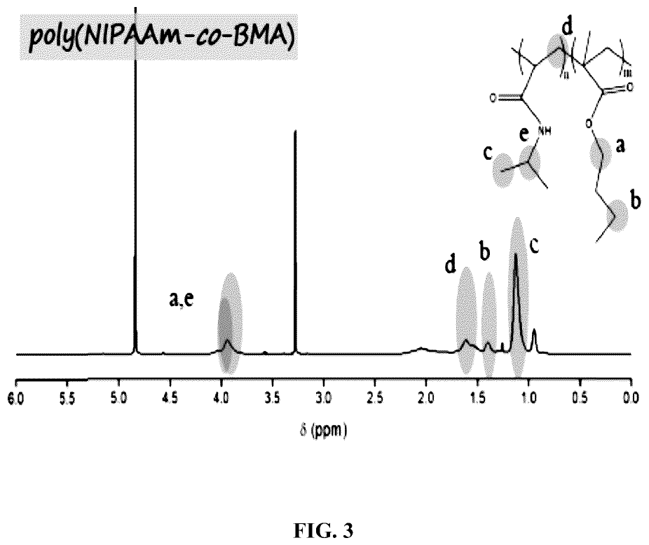

NIPAAm and BMA were dissolved in ethanol and then polymerized in the presence of 4,4′-azobis(4-cyanovaleric acid) (ACVA) at 70° C. for 24 hours. After purification, poly(NIPAAm-co-BMA) was obtained and analyzed by NMR. The NMR spectrum of poly(NIPAAm-co-BMA) was shown in FIG. 3.

### Thermo-Induced Stimuli-Responsive Membranes Coated by Poly(AA-Co-BMA)

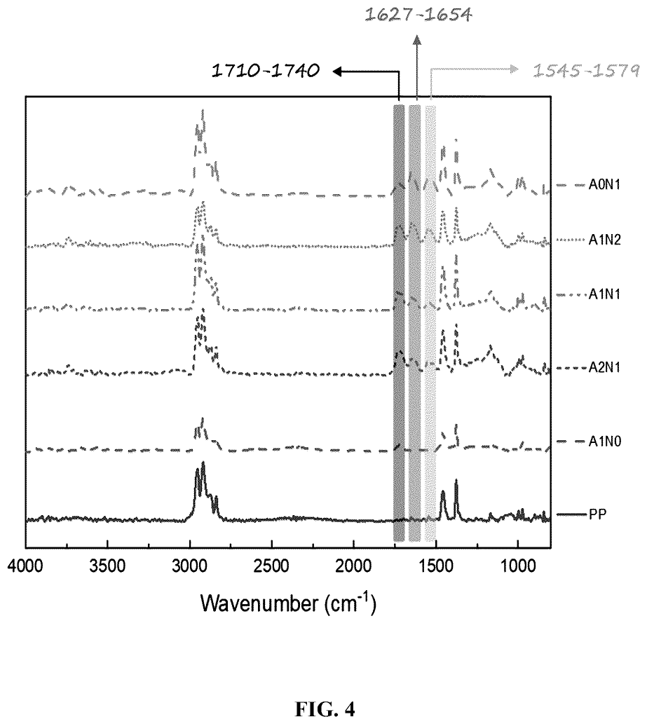

Poly(AA-co-BMA) synthesized from different molar ratio of each monomer was coated on PP to obtained the thermo-induced stimuli-responsive membranes. Composition and functional groups of the membrane surface were characterized by FTIR as shown in FIG. 4 and XPS, and the results were summarized in table 1 and table 2.

TABLE 1

| | | |
|---|---|---|
| Item | Molar ratio | Composition (%) by XPS |

| Copolymer ID | AA | BMA | C | O | C/O |
|---|---|---|---|---|---|
| A2 | 200 | 60 | 79.58 | 20.42 | 3.90 |
| A3 | 300 | 60 | 88.27 | 11.73 | 7.53 |
| A4 | 400 | 60 | 89.32 | 10.68 | 8.36 |
| A5 | 500 | 60 | 90.91 | 9.09 | 10.00 |
| A6 | 600 | 60 | 92.08 | 7.92 | 11.63 |
| PP | / | / | 99.29 | 0.71 | / |

TABLE 2

| Functional group | Binding energy (eV) |
|---|---|
| C—C | 285 |
| O═C—O | 289.26 |
| C═O | 532.22 |
| C—O | 533.52 |

The PP membranes coated by the poly(AA-co-BMA) were measured their coating density directly (non-washed) or after washing. The coating density was calculated according to following equation and summarized in table 3. Herein, W₀ represents original membrane weight and W₁ represents non-washed membrane weight or washed membrane weight after dip coating.

$$Coating desnity ( \frac{mg}{{cm}^{2}} ) = \frac{{W}_{1} - {W}_{0}}{membrane area} . \qquad Equation$$

TABLE 3

| | |
|---|---|
| PP Membrane | Coating density (mg/cm²) |

| poly(AA-co-BMA) | Non-washed | Washed |
|---|---|---|
| A2 | 0.07911 ± 0.023 | 0.02825 ± 0.019 |
| A3 | 0.10359 ± 0.026 | 0.05462 ± 0.016 |
| A4 | 0.09041 ± 0.013 | 0.04709 ± 0.024 |
| A5 | 0.08664 ± 0.026 | 0.03767 ± 0.015 |
| A6 | 0.05839 ± 0.028 | 0.02449 ± 0.012 |

### Thermo-Induced Stimuli-Responsive Membranes Coated by Poly(NIPAAm-Co-BMA)

Poly(NIPAAm-co-BMA) synthesized from different molar ratio of each monomer were coated on PP to obtained the membranes. Composition and functional groups of the membrane surface were characterized by FTIR as shown in FIG. 4 and XPS, and the results were summarized in table 4 and table 5.

TABLE 4

| Item | Molar ratio | | Composition (%) by XPS | | | | |
|---|---|---|---|---|---|---|---|
| Copolymer ID | NIPAAm | BMA | C | O | N | C/O | C/N |
| N1 | 140 | 60 | 79.22 | 13.05 | 7.73 | 6.07 | 10.25 |
| N2 | 200 | 60 | 79.49 | 12.89 | 7.62 | 6.17 | 10.43 |
| N3 | 300 | 60 | 80.74 | 12.60 | 6.61 | 6.41 | 12.21 |
| N4 | 400 | 60 | 83.96 | 10.65 | 5.38 | 7.88 | 15.61 |
| N5 | 500 | 60 | 88.74 | 7.48 | 3.77 | 11.86 | 23.54 |
| N6 | 600 | 60 | 89.54 | 6.85 | 3.61 | 12.63 | 24.80 |
| PP | / | / | 99.29 | 0.71 | 0 | / | / |

TABLE 5

| Functional group | Binding energy (eV) |
|---|---|
| C—C | 285 |
| C—N | 285.94 |
| N—C═O | 288.11 |
| O═C—O | 289.26 |
| C═O | 532.22 |
| C—O | 533.52 |

The PP membranes coated by poly(NIPAAm-co-BMA) were measured their coating density directly (non-washed) or after washing. The coating density was calculated according to the aforementioned equation and summarized in table 6.

TABLE 6

| | |
|---|---|
| Membranes | Coating density (mg/cm²) |

| poly(NIPAAm-co-BMA) | Non-washed | Washed |
|---|---|---|
| N1 | 0.10548 ± 0.030 | 0.02637 ± 0.027 |
| N2 | 0.11866 ± 0.026 | 0.06969 ± 0.042 |
| N3 | 0.13749 ± 0.029 | 0.07534 ± 0.029 |
| N4 | 0.12996 ± 0.050 | 0.08852 ± 0.027 |
| N5 | 0.12054 ± 0.042 | 0.10548 ± 0.030 |
| N6 | 0.07911 ± 0.048 | 0.06404 ± 0.028 |

### Thermo-Induced Stimuli-Responsive Membranes Coated by Copolymer Mixtures

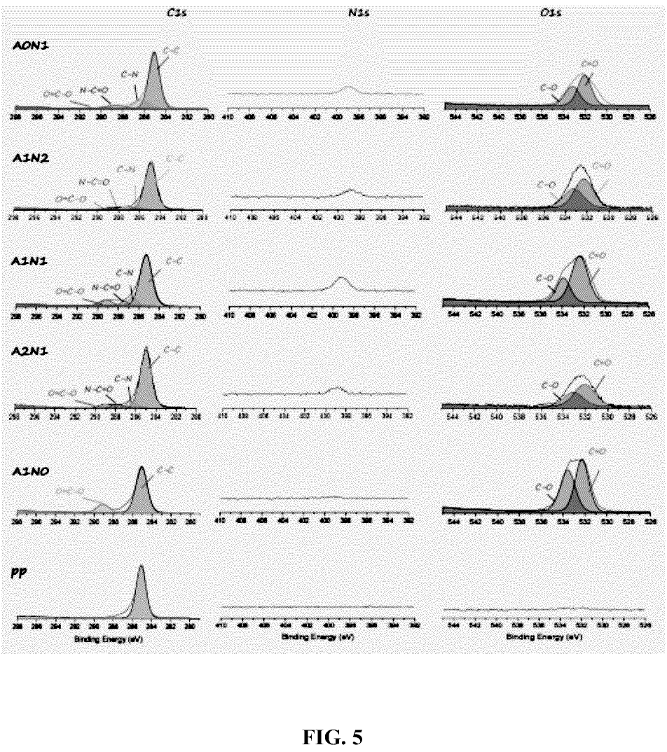

Copolymer mixtures containing different weight ratio of poly(AA-co-BMA) to poly(NIPAAm-co-BMA) were coated on PP membranes. Composition and functional groups of the membrane surface were characterized by FTIR and XPS. The FTIR spectrums are shown in FIG. 4. The XPS spectrums and results were shown in FIG. 5 and summarized in table 7 and table 8.

TABLE 7

| | Weight ratio | | | | | | |
|---|---|---|---|---|---|---|---|
| Item | poly | poly | | | | | |
| Copolymer | (AA-co- | (NIPAAm- | Composition (%) by XPS | | | | |
| Mixture ID | BMA) | co-BMA) | C | O | N | C/O | C/N |
| A0N1 | / | 1 | 77.69 | 12.32 | 10 | 6.31 | 7.77 |
| A1N2 | 1 | 2 | 86.49 | 10.16 | 3.35 | 8.51 | 25.82 |
| A1N1 | 1 | 1 | 84.09 | 12.61 | 3.3 | 6.67 | 25.48 |
| A2N1 | 2 | 1 | 82.48 | 14.9 | 2.63 | 5.54 | 31.36 |
| A1N0 | / | / | 95.72 | 4.28 | 0 | 22.36 | / |
| PP | / | / | 97.81 | 2.19 | 0 | 44.66 | / |

TABLE 8

| Functional group | Binding energy (eV) |
|---|---|
| C—C | 285 |
| C—N | 285.94 |
| N—C═O | 288.11 |
| O═C—O | 289.26 |
| C═O | 532.22 |
| C—O | 533.52 |

### Water Contact Angle Measurement

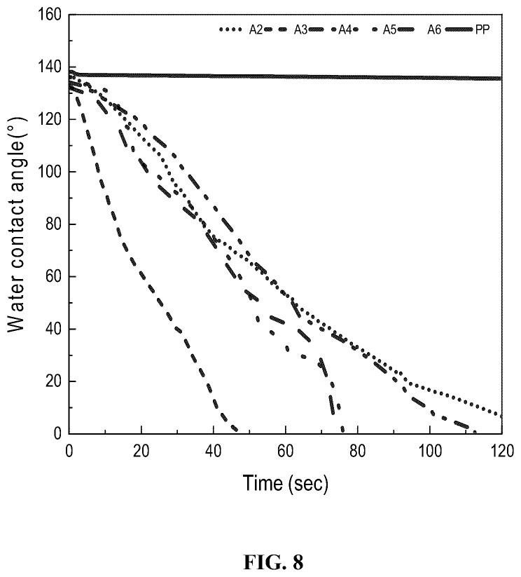

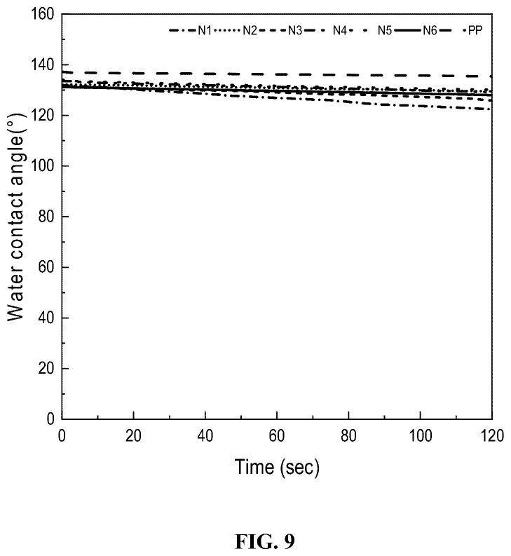

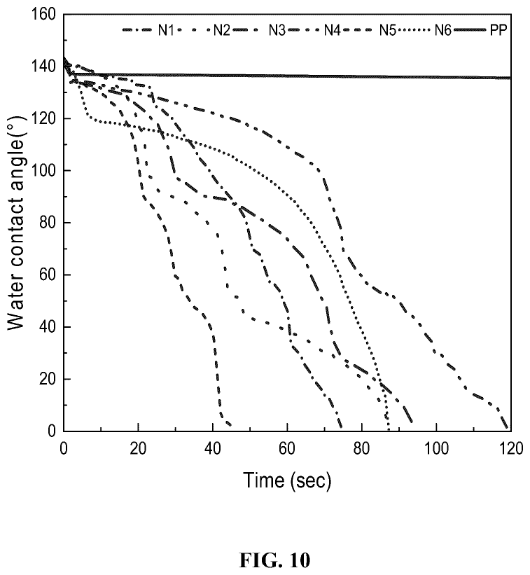

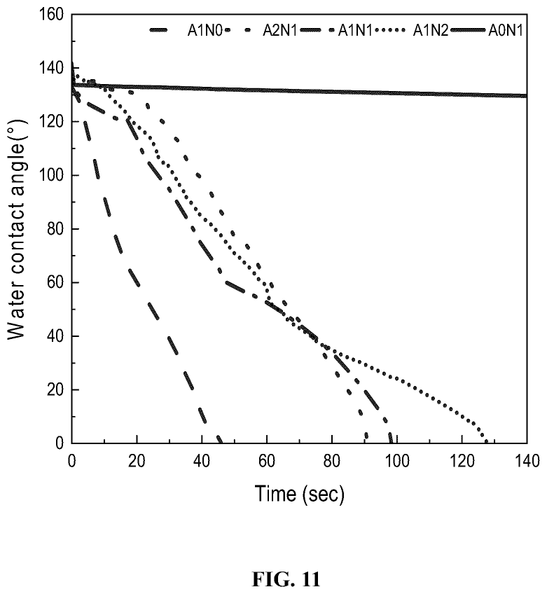

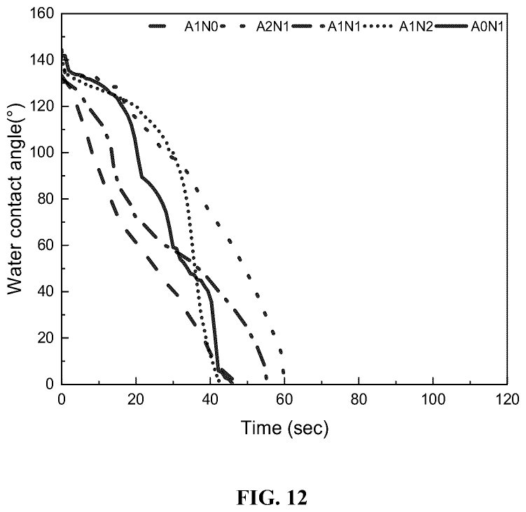

The membranes coated by poly(AA-co-BMA), poly(NIPAAm-co-BMA) or their mixtures were measured water contact angle at different temperature. Please refer to FIG. 8, the membranes coated by poly(AA-co-BMA) show their water contact angle decrease from about 140 degree to zero degree within 120 seconds at 25° C. Please refer to FIG. 9 and FIG. 10, the membranes coated by poly(NIPAAm-co-BMA) show their water contact angle keep constant at 37° C., but decrease from about 135 degree to zero degree within 120 seconds at 4° C. Therefore, the membranes coated by poly(NIPAAm-co-BMA) has a temperature-response surface. Furthermore, the membranes coated by mixtures containing poly(AA-co-BMA) and poly(NIPAAm-co-BMA) also have a temperature-response surface according to FIG. 11 and FIG. 12. Obviously, the water contact angle of the membranes coated by the mixtures decreases more quickly to zero degree within 60 seconds at 4° C. Accordingly, the membranes coated by mixtures containing poly(AA-co-BMA) and poly(NIPAAm-co-BMA) are suitable for capturing or releasing biomaterial at different temperature because they have more sensitive temperature-response surface.

### White Blood Cells Adhesion

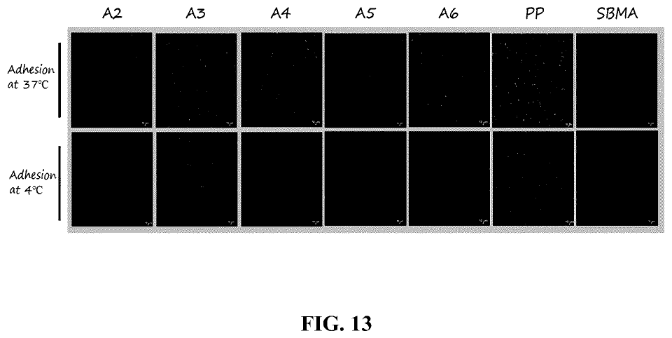

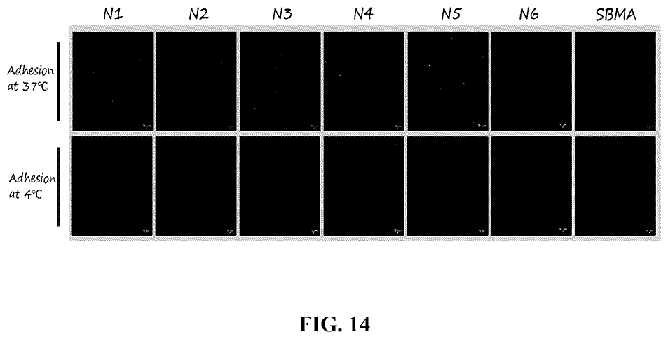

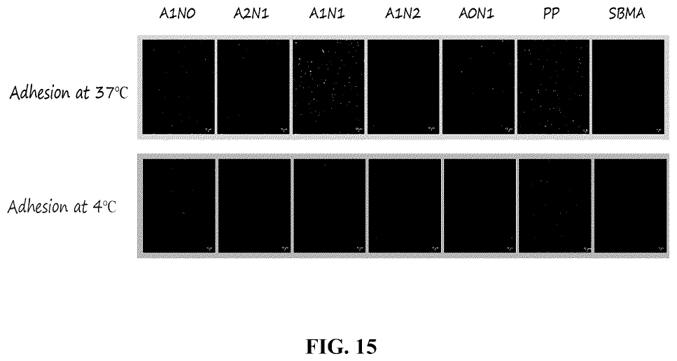

The white blood cells adhesion experiment was conducted by placing the copolymers in a 24-well culture plate, adding 1 mL of phosphate buffered solution (PBS) in each well, and then placing in a 37° C. oven or 4° C. refrigerator for 1 hr. The PBS solution was taken out and added with 1 mL of whole blood samples or white blood cells concentrate. The solution was then placed in a 37° C. oven or 4° C. refrigerator for another 2 hours and the sample was sucked out from the solution. PBS was used to wash out sample that do not adsorb. 1 mL of 2.5% glutaraldehyde solution was added and the solution was set for 24 hours. Finally, a confocal laser scanning microscopy (CLSM) was used to observe the white blood cells adhesion. The images were shown in FIG. 13, FIG. 14, FIG. 15 and FIC. 16. Experimental results were summarized in following tables.

Please refer to FIG. 13 and table 9, it is clear that control group PSBMA does not attach or capture the white blood cells. The copolymer poly(AA-co-BMA) was able to attach or capture the white blood cells at 37° C. and release a small amount of them at 4° C.

TABLE 9

| | Adhesion after cell | Adhesion after cell |
|---|---|---|
| Copolymer | incubation at 37° C. | release at 4° C. |
| ID | (Cells/mm²) | (Cells/mm²) |
| A2 | 678.91 ± 83.17 | 571.45 ± 37.39 |
| A3 | 1350.92 ± 148.31 | 1262.61 ± 56.24 |
| A4 | 1243.46 ± 207.47 | 1010.95 ± 41.86 |
| A5 | 972.67 ± 244.98 | 798.82 ± 35.24 |
| A6 | 795.84 ± 44.68 | 715.55 ± 18.39 |
| PP | 1202.39 ± 89.58 | 1078.88 ± 45.36 |
| SBMA | 2.47026 ± 0 | 0 ± 0 |

Please refer to FIG. 14 and table 10, it is clear that control group PSBMA does not attach or capture the white blood cells. The copolymer poly(NIPAAm-co-BMA) was able to attach or capture the white blood cells at 37° C., and release or detach a large amount of them at 4° C. As a result, poly(NIPAAm-co-BMA) is a good thermo-responsive copolymer.

TABLE 10

| | Adhesion after cell | Adhesion after cell |
|---|---|---|
| Copolymer | incubation at 37° C. | release at 4° C. |
| ID | (Cells/mm²) | (Cells/mm²) |
| N1 | 90.16 ± 17.08 | 70.71 ± 23.93 |
| N2 | 176.93 ± 33.95 | 29.95 ± 6.51 |
| N3 | 201.01 ± 26.75 | 40.76 ± 7.87 |
| N4 | 318.05 ± 55.65 | 30.26 ± 8.15 |
| N5 | 317.42 ± 44.85 | 20.07 ± 5.19 |
| N6 | 307.13 ± 3.84 | 60.27 ± 1.50 |
| PP | 1202.39 ± 89.58 | 1078.88 ± 45.36 |
| SBMA | 2.47026 ± 0 | 0 ± 0 |

Please refer to FIG. 15 and table 11, it is clear that control group PSBMA does not attach or capture the white blood cells. The copolymer mixtures containing poly(AA-co-BMA) and poly(NIPAAm-co-BMA) were able to attach or capture more the white blood cells at 37° C. than poly(NIPAAm-co-BMA), and release or detach a large amount of them at 4° C. As a result, The copolymer mixtures containing poly(AA-co-BMA) and poly(NIPAAm-co-BMA) were are more suitable for capturing or releasing white blood cells at different temperature because they have more sensitive temperature-response surface.

TABLE 11

| | Adhesion after cell | Adhesion after cell |
|---|---|---|
| Copolymer | incubation at 37° C. | release at 4° C. |
| Mixture ID | (Cells/mm²) | (Cells/mm²) |
| A1N0 | 2139.24 ± 290.47 | 1976.2 ± 142.65 |
| A2N1 | 1784.52 ± 108.92 | 250.98 ± 107.02 |
| A1N1 | 1931.04 ± 218.81 | 150.69 ± 28.55 |
| A1N2 | 1290.54 ± 122.88 | 284.08 ± 43.74 |
| A0N1 | 317.42 ± 44.852 | 20.07 ± 5.19 |
| PP | 1202.39 ± 89.58 | 1078.88 ± 45.36 |
| SBMA | 2.47026 ± 0 | 0 ± 0 |

### General Filtration Procedure for WBC Separation

Firstly, attach the 10 ml syringe to a syringe filter, wherein the syringe filter assembled the invented thermo-induced stimuli-responsive membranes. Place the assembled syringe and filter on top of an open 15 ml collection tube. Collect 10 ml samples of freshly collected blood from donors are treated with CPDA-1 anticoagulant and then allow the treated blood samples to drain, via gravity, through the syringe filter into the collection tube. After filtration, prepare a 25 mm diameter syringe module. Add 5 ml of cold PBS solution maintained at 4° C. to the syringe module. Incubate the syringe module at 4° C. for 30 minutes. After incubation, place the filter with the syringe on a new 50 ml collection tube. Push the total 20 ml cold PBS through the filter to recover cells attached to the membranes. Pipette up and down the syringe module to ensure through elution of leukocytes from the filter. The complete solution was then analyzed with a Complete Blood Count (CBC) instrument (XN-1000, Sysmex).

### Leukocyte Filter Performance Evaluation

The syringe filter assembled 3, 6 and 12 layers of the thermo-induced stimuli-responsive membrane were evaluated their performance according to the general filtration procedure for WBC separation, respectively. PP membrane without coating is used as a control group. The experimental results were summarized in table 12 and 13. Depletion, selectivity, recovery and purity are defined by following equations. Herein, WB represents whole blood sample; WBC represent white blood cells; N_{WBC,1} represents before filtration WBC number concentration; N_{WBC,2} represents after filtration WBC number concentration; N_{WBC, 3} represents recovery WBC number concentration; N_{WB, 1} represents recovery WB number concentration N_{WB, 2} represents recovery WB number concentration and N_{WB, 3} represents recovery WB number concentration.

$$Depletion ( \% ) = ( 1 - \frac{{N}_{WBC , 2}}{{N}_{WBC , 1}} ) \times 100. \qquad Depletion$$

$$Selectivity ( \% ) = \frac{{N}_{WBC , 1} - {N}_{WBC , 2}}{{N}_{WB , 1} - {N}_{WB , 2}} \times 100. \qquad Selectivity$$

$$Recovery ( \% ) = \frac{{N}_{WBC , 3}}{{N}_{WB , 1} - {N}_{WB , 2}} \times 100. \qquad Recovery$$

$$Purity ( \% ) = \frac{{N}_{WBC , 1}}{{N}_{WB , 3}} \times 100. \qquad Purity$$

TABLE 12

| | Sample | | | | | | | |
|---|---|---|---|---|---|---|---|---|
| | Whole blood sample | | | | | | WBC concentrates | |
| | Membranes | | | | | | | |
| | 3 layers | | 6 layers | | 12 layers | | 12 layers* | |
| Copolymer | Depletion | Selectivity | Depletion | Selectivity | Depletion | Selectivity | Depletion | Selectivity |
| Mixture ID | % | % | % | % | % | % | % | % |
| A1N0 | 34.66 | 3.18 | 52.19 | 5.49 | 84.99 | 4.92 | 98.98 | 44.67 |
| A2N1 | 18.00 | 2.02 | 46.79 | 3.07 | 79.71 | 11.34 | 95.84 | 38.47 |
| A1N1 | 26.05 | 3.41 | 51.55 | 5.37 | 84.68 | 13.70 | 93.54 | 39.44 |
| A1N2 | 11.70 | 1.47 | 35.58 | 2.65 | 72.57 | 10.84 | 84.15 | 30.34 |
| A0N1 | 20.21 | 1.11 | 49.63 | 1.24 | 78.00 | 1.65 | 99.66 | 4.11 |
| Control | 42.64 | 1.11 | 52.42 | 1.03 | 51.74 | 0.93 | 59.25 | 11.16 |

TABLE 13

| | Sample | | | | | | | |
|---|---|---|---|---|---|---|---|---|
| | Whole blood sample | | | | | | WBC concentrates | |
| | Membranes | | | | | | | |
| | 3 layers | | 6 layers | | 12 layers | | 12 layers* | |
| Copolymer | Recovery | Purity | Recovery | Purity | Recovery | Purity | Recovery | Purity |
| Mixture ID | % | % | % | % | % | % | % | % |
| A1N0 | 12.01 | 1.77 | 10.00 | 2.24 | 6.95 | 2.53 | 3.52 | 0.35 |
| A2N1 | 77.87 | 1.97 | 76.36 | 3.00 | 73.14 | 8.63 | 67.84 | 2.71 |
| A1N1 | 90.88 | 3.79 | 88.71 | 6.39 | 84.46 | 12.12 | 74.55 | 4.53 |
| A1N2 | 73.53 | 1.79 | 71.59 | 2.39 | 67.02 | 7.92 | 69.06 | 2.31 |
| A0N1 | 67.70 | 0.35 | 66.71 | 1.28 | 63.74 | 1.27 | 65.25 | 0.46 |
| Control | 12.01 | 0.13 | 8.51 | 0.12 | 7.64 | 0.07 | 2.13 | 0.81 |

According to table 13 and table 14, the filter assembled 12 layers of the thermo-induced stimuli-responsive membrane show higher recovery and purity. Therefore, the filter assembled 12 layers or more than 12 layers are suitable for recovering white blood cells from whole blood samples.

### WBCs Recovery Efficacy

The filters assembled 26-38 layers of the thermo-induced stimuli-responsive membranes were further evaluated their white blood cells recovery according to the aforementioned filter performance evaluation procedure. The experimental results were summarized in table 14. It is clear that the invented recovering white blood cells method by using the leukocyte filter filtration has a short operation time than known gradient centrifugation method and the total white blood cells recovery on the filter are more than 2×10⁷ cells/ml. Therefore, the invented thermo-induced stimuli-responsive membranes/filters successfully recover the white blood cells from the whole blood sample within 1 hour and show higher WBCs recovery efficacy than current gradient centrifugation method.

TABLE 14

| | | Total WBCs capture on | Total WBCs recovery on | |
|---|---|---|---|---|
| Membrane | Separation | the filter (10 ml whole | the filter (25 ml elution | Operation |
| Surface | method | bloods, WBs) cells/ml | buffer, PBS) cells/ml | time |
| AA | Gravity | 4.31 × 10⁷ | >2.02 × 10⁷ | <1 h |
| single coating | filtration | | | |
| NIPAAm | Gravity | 4.62 × 10⁷ | >2.05 × 10⁷ | <1 h |
| single coating | filtration | | | |
| A85N15 | Gravity | 5.16 × 10⁷ | >2.39 × 10⁷ | <1 h |
| Mixed coating | filtration | | | |
| Ficoll-Paque | Gradient | 10 ml whole bloods | <1 × 10⁶ | >2 h |
| PLUS | Centrifugation | | | |

### Synthesis of Poly(NIPAAm-Co-AA) Hydrogel

Monomers (NIPAAm, AA, or their mixtures), crosslinker, catalyst and initiator were added into water, mixed well to obtain a composition, and the composition was put in a mold for forming a hydrogel. The hydrogel has following structure as shown in formula (1) and was stored in deionized water.

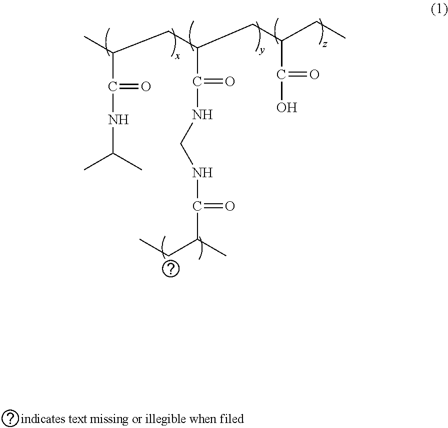

### Properties of the Poly(NIPAAm-Co-AA) Hydrogels

The hydrogels prepared from AA, NIPAAm and different weight ratio of AA to NIPAAm was summarized in table 15. Equilibrium water content was calculated according to following equation. Herein, Wₛ represents weight swelling and W_{d} represents weight dry. The equilibrium water content of the hydrogels are more than 70%, hence, the poly(NIPAAm-co-AA) hydrogels are hydrophilic.

### Equilibrium Water Content

$$Equilibrium water content ( \% ) = ( \frac{{W}_{s} - {W}_{d}}{{W}_{s}} ) \times 100 \%$$

TABLE 15

| | | Equilibrium | | Oil | |
|---|---|---|---|---|---|
| Hydrogel | Weight ratio | water | Error | contact | Error |
| ID | (AA/NIPAAm) | content (%) | bar | angle (°) | bar |
| AAc | 100/0 | 75.49 | 1.31 | 125.89 | 4.33 |
| AAc_5NI | 95/5 | 80.55 | 1.42 | 125.83 | 1.62 |
| AAc_7.5NI | 92.5/7.5 | 75.32 | 2.27 | 124.96 | 1.24 |
| AAc_10NI | 90/10 | 85.57 | 2.18 | 128.29 | 1.04 |
| AAc_12.5NI | 87.5/12.5 | 71.24 | 1.78 | 125.48 | 1.15 |
| AAc_15NI | 85/15 | 84.09 | 3.44 | 126.05 | 2.55 |
| AAc_20NI | 80/20 | 74.71 | 2.27 | 122.24 | 1.34 |
| AAc_50NI | 50/50 | 72.53 | 7.37 | 121.83 | 1.44 |
| AAc_90NI | 10/90 | 84.17 | 0.88 | 120.57 | 1.54 |
| NIPAAm | 0/100 | 87.36 | 1.67 | 121.19 | 0.56 |
| SBMA | / | 69.71 | 4.33 | 137.67 | 1.67 |

### White Blood Cells/Leukocytes Attachment

Use white blood cell concentrate to attach the hydrogels for 1 hour at 37° C. Collect unattached white blood cell concentrate, and use DPBS wash the hydrogels interface. After use Glutaraldehyde and DAPI stain cells, stored the hydrogels in DPBS. Finally, use confocal laser scanning microscopy (CLSM) to observe and analyze surface of the poly(NIPAAm-co-AA) hydrogels

### White Blood Cells/Leukocytes Detachment

Use white blood cell concentrate to attach the hydrogels for 1 hour and use DPBS wash the hydrogels interface unattached cells. Add DPBS for 24 hours at 4° C. to detach white blood cells. Collect the detached white blood cells, and use DPBS wash the hydrogels interface. After use glutaraldehyde and DAPI stain cells, stored the hydrogels in DPBS. Finally, use confocal laser scanning microscopy (CLSM) to observe and analyze surface of the poly(NIPAAm-co-AA) hydrogels.

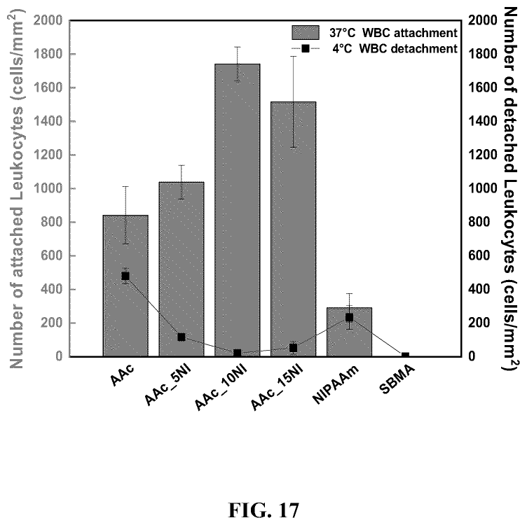

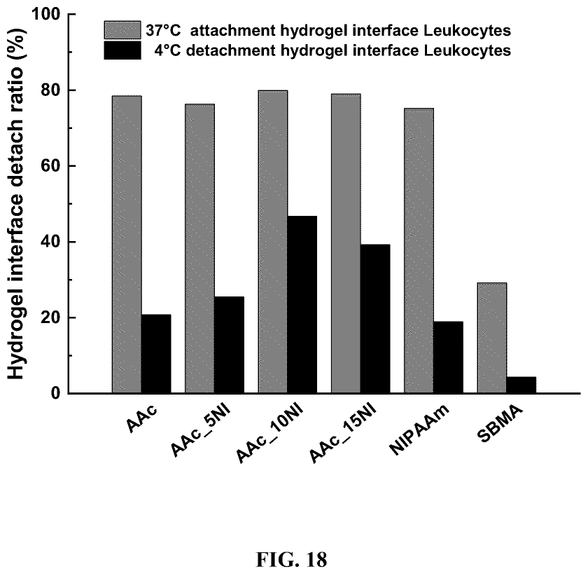

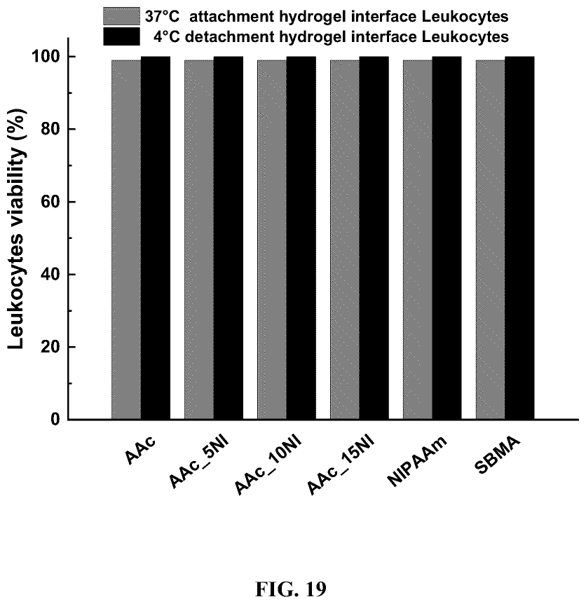

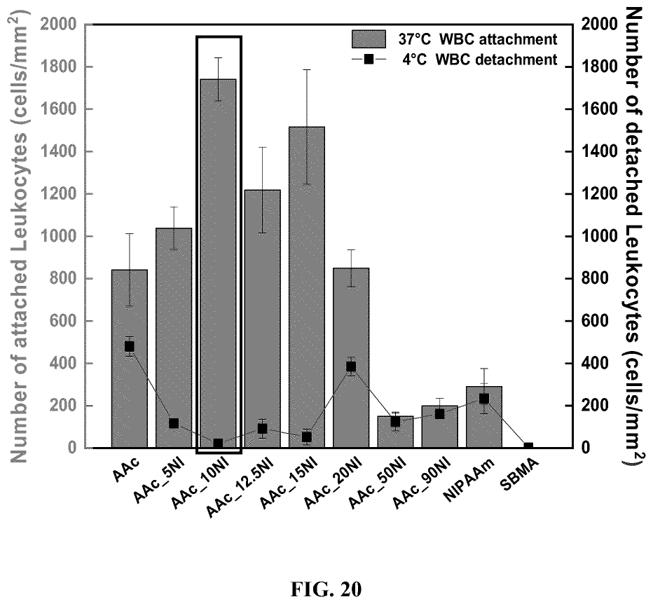

The white blood cells attachment and detachment results are summarized in table 16, table 17 and table 19. Please refer to FIG. 17 FIG. 18 and FIG. 20, the invented poly(NIPAAm-co-AA) hydrogels attach or capture white blood cells at 37° C. and detach or release the white blood cells at 4° C. Control group SBMA does not show any leukocytes attaching behavior. Moreover, according to table 18 and FIG. 19, the poly(NIPAAm-co-AA) hydrogels have more than 99% viability and that means the poly(NIPAAm-co-AA) hydrogels are compatible with the white blood cells. Herein, attachment hydrogel interface Leukocytes (%) and detachment hydrogel interface Leukocytes (%) are calculated according to following equations.

### Equation for Attachment Hydrogel Interface Leukocytes (%)

$$Attachment hydrogel interface leukocyte ( \% ) = \frac{37 ° C . attached leukocytes}{Total leukocytes} \times 100 \%$$

### Equation for Detachment Hydrogel Interface Leukocytes (%)

$$Detachment hydrogel interface leukocyte ( \% ) = \frac{4 ° C . detached leukocytes}{Total leukocytes - 37 ° C . attached leukocytes )} \times 100 \%$$

TABLE 16

| | 37° C. WBC | | 4° C. WBC | |
|---|---|---|---|---|
| Hydrogel | attachment | Error | detachment | Error |
| ID | (Cells/mm²) | bar | (Cells/mm²) | bar |
| AAc | 841.33 | 170.89 | 479.66 | 46.70 |
| AAc_5NI | 1038.33 | 100.66 | 116.58 | 12.51 |
| AAc_10NI | 1741 | 101.61 | 21 | 8 |
| AAc_15NI | 1515.66 | 270.36 | 52.33 | 37.43 |
| NIPAAm | 291.12 | 84.57 | 233.85 | 71.48 |
| SBMA | 1 | 0 | 2.47 | 0 |

TABLE 17

| Hydrogel | 37° C. attachment hydrogel | 4° C. detachment hydrogel |
|---|---|---|
| ID | interface Leukocytes (%) | interface Leukocytes (%) |
| AAc | 78.476 | 20.775 |
| AAc_5NI | 76.315 | 25.506 |
| AAc_10NI | 79.898 | 46.726 |
| AAc_15NI | 79.008 | 39.227 |
| NIPAAm | 75.195 | 18.908 |
| SBMA | 29.195 | 4.335 |

TABLE 18

| | 37° C. attachment | 4° C. detachment |
|---|---|---|
| Hydrogel | hydrogel interface | hydrogel interface |
| ID | Leukocytes viability(%) | Leukocytes viability (%) |
| AAc | 99.00011 | 99.99987 |
| AAc_5NI | 99.00088 | 99.99996 |
| AAc_10NI | 99.00006 | 99.99988 |
| AAc_15NI | 99.00006 | 99.99988 |
| NIPAAm | 99.00011 | 99.99999 |
| SBMA | 99 | 100 |

TABLE 19

| | 37° C. WBC | | 4° C. WBC | |
|---|---|---|---|---|
| Hydrogel | attachment | Error | detachment | Error |
| ID | (Cells/mm²) | bar | (Cells/mm²) | bar |
| AAc | 841.33 | 170.89 | 479.66 | 46.70 |
| AAc_5NI | 1038.33 | 100.66 | 116.58 | 12.51 |
| AAc_10NI | 1741 | 101.61 | 21 | 8 |
| AAc_12.5NI | 1218 | 202.40 | 91.66 | 44.79 |
| AAc_15NI | 1515.66 | 270.36 | 52.333 | 37.43 |
| AAc_20NI | 848.94 | 86.92 | 384.53 | 44.06 |
| AAc_50NI | 150.66 | 19.73 | 123.66 | 42.35 |
| AAc_90NI | 199.26 | 36.67 | 160.56 | 7.54 |
| NIPAAm | 291.12 | 84.57 | 233.85 | 71.48 |
| SBMA | 1 | 0 | 2.47 | 0 |

Obviously, many modifications and variations are possible in the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A thermo-induced stimuli-responsive membrane, comprising a layer coated on a porous substrate, and composition of the layer comprises at least one copolymer selected from one of group consisting of poly(acrylic acid-co-alkyl methacrylate), poly(N-alkyl acrylamide-co-alkyl methacrylate) and their mixture.

2. The thermo-induced stimuli-responsive membrane of claim 1, having a coating density more than 0.02 mg/cm².

3. The thermo-induced stimuli-responsive membrane of claim 1, wherein the layer has characteristic peaks in XPS spectrum at following binding energy: 285±0.2 eV, 285.94±0.2 eV, 288.11±0.2 eV, 289.26±0.2 eV, 532.22±0.2 eV and 533.52±0.2 eV.

4. The thermo-induced stimuli-responsive membrane of claim 1, wherein the porous substrate comprises PP, PTFE, PVDF, PET, PBT, PU, nylon, PE, PS, ceramic or rayon.

5. The thermo-induced stimuli-responsive membrane of claim 1, wherein the poly(acrylic acid-co-alkyl methacrylate) has a molar ratio of acrylic acid to alkyl methacrylate being from 1.1 to 5.

6. The thermo-induced stimuli-responsive membrane of claim 1, wherein the poly(acrylic acid-co-alkyl methacrylate) has a weight average molecule weight between 1 and 300 kDa.

7. The thermo-induced stimuli-responsive membrane of claim 1, wherein the poly(N-alkyl acrylamide-co-alkyl methacrylate) has a molar ratio of N-alkyl acrylamide to alkyl methacrylate being from 1 to 6.

8. The thermo-induced stimuli-responsive membrane of claim 1, wherein the poly(N-alkyl acrylamide-co-alkyl methacrylate) has a weight average molecule weight between 1 and 300 kDa.

9. The thermo-induced stimuli-responsive membrane of claim 1, wherein the mixture comprises 1-99 wt. % of poly(acrylic acid-co-butyl methacrylate) and 1-99 wt. % of poly(N-isopropylacrylamide-co-butyl methacrylate).

10. A method for recovering white blood cells, comprising,
filtering a blood sample comprises white blood cells through a filter that comprises at least 12 layers of a thermo-induced stimuli-responsive membrane or a column that comprises a leukocyte separation bed by gravity at 25-40° C. for attaching the white blood cells from the blood sample onto the thermo-induced stimuli-responsive membrane or the leukocyte separation bed;
incubating the filter or column at 0-10° C. for 10 minutes at least for detaching the white blood cells from the thermo-induced stimuli-responsive membrane or the leukocyte separation bed; and
eluting the filter or column with a liquid by gravity at 0-10° C. to recover the white blood cells from the blood sample and obtain a white blood cells concentrate that has a concentration of the white blood cells more than 2.0×10⁷ cells/ml.

11. The method of claim 10, wherein the thermo-induced stimuli-responsive membrane comprises a layer coated on a porous substrate, and composition of the layer comprises at least one copolymer selected from one of group consisting of poly(acrylic acid-co-alkyl methacrylate), poly(N-alkyl acrylamide-co-alkyl methacrylate) and their mixture.

12. The method of claim 10, wherein the thermo-induced stimuli-responsive membrane has a coating density more than 0.02 mg/cm².

13. The method of claim 11, wherein the layer has characteristic peaks in XPS spectrum at following binding energy: 285±0.2 eV, 285.94±0.2 eV, 288.11±0.2 eV, 289.26±0.2 eV, 532.22±0.2 eV and 533.52±0.2 eV.

14. The method of claim 11, wherein the porous substrate comprises PP, PTFE, PVDF, PET, PBT, PU, nylon, PE, PS, ceramic or rayon.

15. The method of claim 11, wherein the poly(acrylic acid-co-alkyl methacrylate) has a molar ratio of acrylic acid to alkyl methacrylate being from 1.1 to 5.

16. The method of claim 11, wherein the poly(acrylic acid-co-alkyl methacrylate) has a weight average molecule weight between 1 and 300 kDa.

17. The method of claim 11, wherein the poly(N-alkyl acrylamide-co-alkyl methacrylate) has a molar ratio of N-alkyl acrylamide to alkyl methacrylate being from 1 to 6.

18. The method of claim 11, wherein the poly(N-alkyl acrylamide-co-alkyl methacrylate) has a weight average molecule weight between 1 and 300 kDa.

19. The method of claim 10, wherein the leukocyte separation bed formed from poly(acrylic acid) hydrogel, poly(N-isopropylacrylamide) hydrogel or a hydrogel synthesized from a mixture of acrylic acid and N-isopropylacrylamide.

20. The method of claim 19, wherein the mixture of acrylic acid and N-isopropylacrylamide has a weight ratio of acrylic acid to N-isopropylacrylamide being 4-9.
